(19) United States
(12) Patent Application Publication
(10) Pub. No.: US 2006/0015956 A1
(43) Pub. Date: Jan. 19, 2006

(54) Method to decrease the rate of polyspermy in IVF

(75) Inventor: Randall S. Prather, Rocheport, MO (US)

Correspondence Address: SENNIGER POWERS, ONE METROPOLITAN SQUARE, 16TH FLOOR, ST LOUIS, MO, 63102, US

(73) Assignee: The Curators of the University of Missouri, Columbia, MO (US)

(21) Appl. No.: 11/159,758

(22) Filed: Jun. 23, 2005

Related U.S. Application Data
(60) Provisional application No. 60/620,839, filed on Oct. 21, 2004.
(60) Provisional application No. 60/583,293, filed on Jun. 25, 2004.

(51) Int. Cl. A01K 67/027

(52) U.S. Cl. 800/17; 800/21

(57) ABSTRACT

The field of invention generally relates to increasing the efficiency of in vitro fertilization by decreasing the rate of polyspermy. One aspect of the invention provides a method of reducing polyspermy in in vitro fertilization by forming an in vitro fertilization mixture that contains osteopontin, oocytes, and sperm, and allowing fertilization of the oocyte by sperm. Another aspect of the invention provides an aqueous mixture for in vitro fertilization that contains osteopontin, oocytes, and sperm.

This application claims priority to U.S. provisional patent application Ser. No. 60/620,839 filed Oct. 21, 2004 and U.S. provisional patent application Ser. No. 60/583,293 filed Jun. 25, 2004, both of which are incorporated herein by reference.

### FIELD OF THE INVENTION

The present invention generally relates to increasing the efficiency of in vitro fertilization by decreasing the rate of polyspermy.

### BACKGROUND

Although embryos produced by in vitro maturation (IVM)/in vitro fertilization (IVF) develop to the blastocyst stage, a high incidence (often exceeding 50%) of polyspermy remains a major impediment to the development of efficient systems of IVF in pig. Wang et al., J Reprod Fertil (1997) 111, 101-108. Polyspermic fertilization occurs less frequently in vivo than in vitro in the pig, with the incidence of polyspermy in vivo often less than 5%. Hunter, Mol Reprod Dev (1991), 29:385-391. Polypronuclei can participate in karyosyngamy and the resulting polyploid eggs can develop into diploid, triploid, or mosaic fetuses (Xia, Microscopy Research and Technique (2003), 61, 325-326) that would have difficulty in completing gestation. Polyspermic fertilization occurs more frequently in the pig than in the other species, even for in vivo fertilization under diverse experimental conditions. Hunter, J Reprod Fertil (1967) 13, 133-147; Hunter, J Reprod Fertil (1990) 40, 211-226; Hunter, Mol Reprod Dev (1991) 29, 385-391.

Various approaches have been employed in attempts to overcome the problem of polyspermic fertilization. See generally Funahashi, Reprod Fert Dev (2003) 15, 167-177. Some researchers have focused on the type of IVF medium and certain modifications to that medium in an attempt to mimic in vivo conditions in the oviducts. For example, researchers have co-cultured spermatozoa with oviduct cells (Nagai and Moor, Mol Reprod Dev (1990) 26, 377-382), follicle cells (Wang et al., J Reprod Dev (1992) 38, 125-131), oviductal fluid (Kim et al., J Reprod Fertil (1996) 107, 79-86), follicular fluid (Funahashi and Day, J Reprod Fertil (1993) 99, 97-103), and other substances (Funahashi et al., Biol Reprod (2000) 63, 1157-1163). While reducing sperm number during IVF decreased polyspermic penetration, it also reduced sperm penetration rates. Abeydeera and Day, Biol Reprod (1997) 57, 729-734. But in the approaches listed above, reduction of polyspermic penetration generally came at the cost of an overall reduction in the efficiency of fertilization. In addition, undefined biologicals (such as co-culture with oviduct cells, or addition of follicular fluid, or oviductal fluid) are unstable factors, and these results are not readily repeatable. Li et al., Biol Reprod (2003) 69, 1580-1585. Other suggested approaches include use of embryo cryopreservation straws rather than microdrops (Li et al., Biol Reprod (2003) 69, 1580-1585) and controlling sperm-zona binding (Funahashi, Reprod Fert Dev (2003) 15, 167-177).

Several researchers have focused upon the problem of polyspermy specifically in pig. Pig oocytes flushed from the oviduct on Day 2 of the estrous cycle and subsequently fertilized in vitro have been observed to have a much lower incidence of polyspermy (28%) than oocytes matured and fertilized in vitro (62%). Wang et al., Mol Reprod Dev (1998) 49:308-316. Other pig-specific attempted solutions to the problem of IVF polyspermy include use of periovulatory oviduct-conditioned media (Vatzias and Hagen, Biol Reprod (1999) 60, 42-48), oviduct fluid (Funahashi and Day, J Reprod Fertil (1993) 99, 97-1038; Kim et al., Zygote (1997) 5, 61-65), and coincubation of boar spermatozoa or pig oocytes with oviductal epithelial cells (Nagai and Moor, Mol Reprod Dev (1990) 26, 377-382; Kano et al., Theriogenology (1994) 42, 1061-1068; Dubuc and Sirard, Mol Reprod Dev (1995) 41, 360-367).

Osteopontin is an extracellular matrix protein; it is an acidic single chain phosphorylated glycoprotein component. In general, osteopontin is a monomer ranging in length from 264-301 amino acids that undergoes extensive post-translational modification, including phosphorylation, glycosylation, and cleavage resulting in molecular weight variants ranging from 25-75 kDa. Johnson et al., Biol Reprod (2003) 69, 1458-1471. Among several reported functions, osteopontin has been reported to be involved with mammalian reproductive systems. Johnson et al., Biol Reprod (2003) 69, 1458-1471; Garlow et al., Biol Reprod (2002) 66, 718-725. One researcher has reported that treating bovine oocytes with purified bovine milk osteopontin increased the rate of cleavage and embryonic development in vitro. Goncalves et al., Soc for Study of Reprod (2003) 68 supp. 1, 336-337.

### SUMMARY OF THE INVENTION

Among the various aspects of the present invention may be noted a process for in vitro fertilization with a lower incidence of polyspermic fertilization. The process and associated compositions are particularly advantageous in connection with the in vitro fertilization of swine. Briefly, therefore, the present invention is directed to compositions and a process for reducing polyspermy in the production of embryos. The process comprises forming a mixture containing an anti-polyspermy agent, oocytes, and sperm and allowing the sperm to fertilize the oocyte. The composition contains osteopontin, oocytes, and sperm.

Other objects and features will be in part apparent and in part pointed out hereinafter.

### BRIEF DESCRIPTION OF THE DRAWINGS

FIG. 1 is an image of the acrosome reaction on the surface of the zona pellucida as observed with an epi-fluorescent microscope at 1000× magnification. Letters “a” and “e” designate spermatozoa with a reacted acrosome. Letter “b” designates a spermatozoa with an intact acrosome. Letters “c” and “d” designate spermatozoa without an acrosome. FIG. 1A shows the DNA staining. FIG. 1B shows the acrosomal staining. FIG. 1C shows the merged images. Numeral “1” designates the acrosomal region of spermatozoon. Numeral “2” designates the nuclear region of spermatozoon. Methodology is as described in Example 5.

### DETAILED DESCRIPTION OF THE INVENTION

Surprisingly, it has been discovered that the incidence of polyspermy during the production of embryos can be reduced during in vitro fertilization by the use of osteopontin and equivalent anti-polyspermy agents during in vitro fertilization procedures. While such anti-polyspermy agents can generally be used during in vitro fertilization of a range of species, e.g., porcine, human, bovine, canine, equine, ovine, avian, and rodent, they offer particular advantages during porcine in vitro fertilizations in which there tends to be a greater incidence of polyspermy.

The process of the present invention comprises forming an in vitro fertilization mixture containing the anti-polyspermy agent, an oocyte, and sperm, and allowing the sperm to fertilize the oocyte. In vitro fertilization processes are well known; see e.g. Fan and Sun, Methods in Molecular Biology, vol. 253, Germ Cell Protocols, vol. 1 Sperm and Oocyte Analysis, Ed. Shatten, Humana Press Inc., Totowa, N.J. (2004) 227-233. Except as otherwise noted herein, therefore, the process of the present invention is carried out in accordance with any such processes.

In general, the anti-polyspermy agent is osteopontin or an analog or mimic thereof. Osteopontin contains the conserved Arg-Gly-Asp (RGD) sequence, which is known to interact with cell surface receptors. Osteopontin also contains over twenty conserved phosphoacceptor serine residues, generally localized in Ser/Thr-X-Glu/Ser(P)/Asp or Ser-X-X-Glu/Ser(P) motifs. Preferably, the osteopontin is a purified osteopontin. Wild-type osteopontin can be obtained as described in, for example, McFarland et al., Annals New York Acad Sciences (1995) 760, 327-331. Mutant osteopontin can be obtained as described in, for example, Johnson et al., Biol Reprod. (2001) 65, 820-828.

The concentration of the anti-polyspermy agent will typically be in the range of about 0.001 to about 1.0 micrograms per milliliter of fertilization mixture. For example, when the anti-polyspermy agent is osteopontin, the concentration is preferably in the range of about 0.01 to about 0.1 μg/ml. In addition, while the anti-polyspermy agent can be immobilized to beads or other solid support (e.g., the interior surface of the container holding the in vitro fertilization mixture), it is generally preferred that the agent be dissolved in the in vitro fertilization mixture.

In the absence of an anti-polyspermy agent, the polyspermy rate (number of oocytes with >1 sperm/total number of oocytes penetrated) in pig is typically greater than about 40%, often exceeding about 50%. In one embodiment of the present invention, the addition of osteopontin reduces the rate of polyspermy to less than about 36%. For example, osteopontin can reduce the rate of polyspermy to less than about 33%, less than about 30%, less than about 27%, less than about 25%, less than about 23%, less than about 20%, less than about 18%, less than about 16%, or less than about 14% (see e.g. Example 4; Table 1).

Oocyte Mixture

In one embodiment, the in vitro fertilization mixture is formed by combining sperm with a pre-formed oocyte mixture containing at least one oocyte, the anti-polyspermy agent, and optionally one or more additives. Typically, an oocyte mixture is formed by combining a buffer appropriate for IVM or IVF with one or more oocytes, the anti-polyspermy agent, and one or more additives, for example, a metabolite such as pyruvate, a sugar such as glucose or sorbitol, caffeine, an enzyme such as hyaluronidase, an antibiotic such as gentamicin, penicillin, or streptomycin, or an amino acid or amino acid analog such as cysteine, glutamine, taurine, or hypotaurine. Other suitable additives are described in further detail in, for example, Fan and Sun (2004). In a preferred embodiment, the buffer is a modified TCM 199 buffer (see e.g. Example 1). In another embodiment, the pre-formed oocyte mixture contains at least one oocyte, a buffer, and one or more additives, wherein the osteopontin is added to the in vitro fertilization mixture either simultaneously with the oocyte mixture and the sperm mixture, or as a component of the sperm mixture.

Oocyte(s) to be included in the oocyte mixture can be obtained commercially (e.g., BoMed, Madison, Wis.) or collected directly from a female. As an example, oocytes can be collected as cumulus-oocyte complexes and matured in a suitable in vitro oocyte maturation medium (see e.g. Examples 1, 2). Procedures for IVM of oocytes from porcine follicles to acquire meiotic competence and capacity to be fertilized are described in, for example, Abeydeera et al., Biol. Reprod. (1998) 58, 1316-1320; and Abeydeera et al., Zygote (2001) 9, 331-337. As an example, approximately 25-100 cumulus-oocyte complexes can be matured in approximately 500 μl of in vitro maturation medium covered with mineral oil (see e.g. Example 2). Oocyte maturation can occur from about 37° C. to about 40° C. Preferably, oocyte maturation will occur at about the body temperature of the subject animal. For example, in porcine, oocyte IVM can be carried out at about 39° C. Maturated oocytes can then be stripped of the cumulus cells and suspended in a suitable IVF medium, such as a modified Tris-buffered medium, as described in Example 1 or Fan and Sun (2004). Whether or not osteopontin is present, after oocytes are matured, they can be transferred into droplets of a medium suitable for IVF. The fertilization droplets containing oocytes can be, for example, approximately 50 μl, covered in mineral oil, and equilibrated 40-44 hours at 38.5° C. in 5% CO₂ in air (see e.g. Examples 2, 3).

Osteopontin or another anti-polyspermy agent can be introduced to the oocyte mixture at any point during the above described procedures. For example, osteopontin can be added at a concentration of about 0.001 to about 1.0 μg/ml of the final oocyte mixture. In one preferred embodiment, osteopontin is added at a concentration of about 0.01 to about 0.1 μg/ml to the oocyte IVM mixture so as to be present during the maturation process.

The oocyte mixture can optionally be incubated for a period of time before it is combined with sperm to form the IVF mixture. For example, the oocyte mixture can be incubated for a period of up to about 48 hours before being combined with sperm to form the IVF mixture. Preferably the oocyte mixture is incubated for over two hours up to about 48 hours.

Sperm Mixture

Sperm useful to the methods of the invention can be obtained commercially (e.g., Lone Willow USA, Inc., Roanoke, Ill.) or collected directly from a male. Collected sperm can be used directly as a fresh ejaculate or extended, sorted, and/or cryopreserved and used later in accordance with conventional procedures. See e.g. Fan and Sun (2004); Pursel and Johnson, J Anim Sci (1976) 42, 927-931. Cryopreservation can be practiced as described in, for example, Suzuki et al., Microscopy Research & Technique (2003) 61, 327-334. Presorting of sperm to select for X chromosome or Y chromosome bearing sperm can be practiced as described in, for example, Abeydeera et al., Theriogenology (1998) 50, 981-988. The sperm used for fertilization can be used to carry into the oocyte DNA for sperm-mediated transgenesis, as described in, for example, Lavitrano et al., Molecular Reproduction and Development (2003) 64, 284-297.

Regardless of source, the sperm mixture generally contains sperm suspended in a medium. The medium may include seminal fluid, buffer, and/or additives. For example, the medium of the sperm mixture may be exclusively seminal fluid (i.e., neat ejaculate), a mixture of seminal fluid and a buffer, or exclusively buffer. Among other things, the buffer should be non-toxic to the cells and can enhance sperm viability by buffering the sperm suspension against significant changes in pH or osmotic pressure. Exemplary buffers include phosphates, diphosphates, citrates, acetates, lactates, and combinations thereof. Additionally, the sperm mixture may or may not contain an anti-polyspermy agent, for example osteopontin. In one embodiment, the sperm mixture is fresh ejaculate. In another embodiment, the sperm mixture contains sperm, seminal fluid, and osteopontin. In a further embodiment, the sperm mixture contains sperm, a buffer (preferably a buffer suitable for sperm washing, sperm maturation, or IVF), and osteopontin.

Osteopontin or other anti-polyspermy agent can be introduced to the sperm mixture at any point in the previously described steps. For example, osteopontin can be included during washing or resuspension of the cryospreserved sperm mixture. Or, osteopontin can be included in the diluted sperm mixture prior to cryospreservation of the sperm sample. Regardless of the point of introduction, the osteopontin or other anti-polyspermy agent will typically be added at a concentration of about 0.001 to about 1.0 micrograms per milliliter of the final sperm mixture. For example, osteopontin can be added at a concentration of about 0.01 to about 0.1 μg/ml.

Optionally, the sperm mixture can be incubated for a period of time before being combined with an oocyte to form an IVF mixture. For example, the sperm mixture can be incubated up to about 6 hours.

In Vitro Fertilization Mixture

The IVF mixture of the present invention contains sperm, at least one oocyte, and the anti-polyspermy agent. These components can be combined through various routes. For example, a pre-formed oocyte mixture containing the anti-polyspermy agent can be combined with sperm. Alternatively, a pre-formed sperm mixture containing the anti-polyspermy agent can be combined with at least one oocyte. In another alternative approach, a pre-formed oocyte mixture containing the anti-polyspermy agent is combined with a pre-formed sperm mixture containing the anti-polyspermy agent. In yet another alternative approach, the anti-polyspermy agent is introduced into the IVF mixture simultaneously with or subsequent to the introduction of the sperm and oocyte(s) into the mixture. Preferably, a sperm mixture is combined with a droplet of oocyte mixture to form an IVF droplet. As an example, approximately 50 μl of sperm sample can be added to an oocyte droplet, providing a final sperm concentration of about 1×10⁵ cells/ml to about 1×10⁶ cells/ml (see e.g. Example 3).

In Vitro Fertilization

The IVF mixture can be incubated for a period of time after sperm and oocytes are combined to allow fertilization to occur. In one embodiment, the IVF mixture is incubated up to about 6 hours. For example, the IVF mixture can be incubated for up to about 5 hours. As another example, the IVF mixture can be incubated for up to about 1 hour. Typically, fertilization will occur within about one hour. As an example, an IVF droplet containing oocytes, sperm, and osteopontin can be incubated at 38.5° C. in an atmosphere of 5% CO₂ in air and 100% relative humidity (see e.g. Example 3).

The spermatozoa can be removed at the beginning of the fertilized oocyte incubation period or at any time throughout the developmental incubation period. One skilled in the art will recognize that the time of optimal sperm removal is closely correlated to the desired rate of oocyte penetration. Generally, 50% is acceptable penetration, while at least about 80% or at least about 90% is preferred. As an example, the embryos can be harvested at 24 hours to check for the presence of pronuclei and vortexed to remove sperm bound to the zona pellucida. As another example, porcine embryos can be harvested at 18 hours to check for the presence of pronuclei and vortexed to remove sperm bound to the zona pellucida. Removal of loosely attached sperm can be performed, for example, by washing three times in a suitable developmental medium such as NCSU 23 with 0.4% BSA or PZM3 (see e.g. Example 3).

In several embodiments, the addition of osteopontin increases the efficiency of IVF (number of oocytes with 1 male and 1 female pronucleus/total number of oocytes inseminated) without substantially decreasing penetration rate (number of oocytes penetrated by sperm/total number of oocytes inseminated). In vitro fertilization rates are determined by measuring the percent fertilization of oocytes in vitro. At the end of the incubation of sperm and oocytes, oocytes can be stained with an aceto-orcein stain or the equivalent to determine the percent oocytes fertilized. Alternatively, fertilized oocytes can be left in culture for about 2 days, during which division occurs and the number of cleaving embryos (i.e., 2 or more cells) are counted. Nuclear status (pronuclear, sperm head, sperm tail, MII chromosome, Pb1, Pb2) can be assessed by examining the stained oocytes under a phase contrast microscope. See e.g. Abeydeera et al., Biol Reprod (1998) 58:1316-1320. In one embodiment, addition of osteopontin increases the efficiency of IVF to greater than about 35%. For example, addition of osteopontin can increase the efficiency of IVF to greater than about 38%, greater than about 40%, greater than about 42%, greater than about 44%, greater than about 46%, greater than about 48%, or greater than about 50%.

Additives

Various additives can be included in the in vitro fertilization mixture to further reduce the incidence of polyspermy or increase the efficiency of fertilization. For example, porcine oviduct-specific glycoprotein can be included in porcine IVF mixtures; porcine oviduct-specific glycoprotein is known to reduce the incidence of polyspermy in pig oocytes, reduce the number of bound sperm, and increase post-cleavage development to blastocyst. Kouba et al., Biol Reprod (2000) 63, 242-250. According to the methods of the invention, the addition of osteopontin in conjunction with porcine oviduct-specific glycoprotein will further decrease the incidence of polyspermy in porcine IVF.

Such additives can be introduced to the IVF mixture by various routes. For example, the additive can be included in an oocyte mixture which is then combined with sperm to form the IVF mixture, it can be included in a sperm mixture which is combined with an oocyte or oocyte mixture to form the IVF mixture, or it can be added directly to the IVF mixture after sperm and oocyte are combined.

Use of Embryo

In one embodiment, the fertilized oocyte is cultured to produce an embryo. An “embryo” refers to an animal in early stages of growth following fertilization up to the blastocyst stage. The blastocyst stage has two cell types: the inner cell mass cells, which are generally considered totipotent cells; and the trophectoderm cells which are generally considered to be a differentiated epithelial cell layer (or sphere). In contrast, somatic cells of an individual are cells of a body that are differentiated and are not totipotent. After allowing sufficient time for fertilization and subsequent washing, the oocytes are transferred into a suitable development medium and incubated under conditions suitable for further development of fertilized oocytes into embryos. In general, the medium for culturing sperm, oocytes, or embryos will be a balanced salt solution, examples of which include Ml 99, Porcine Zygote Medium-3 (PZM3), Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media (PETS, Canton, Tex.).

As an example, washing, transfer, incubation, and culturing of fertilized oocytes and embryos can be practiced as described in Fan and Sun (2004); and Petters and Wells, J Reprod Fertil (1993) 48, 61-73. As a further example, the oocytes can be washed in a development medium, such as Porcine Zygote Medium with BSA, transferred into 500 μl of the same development medium in a 4-well Nunclon dish, covered with mineral oil (to prevent drying of sample and alteration of osmolarity) and incubated at 38.5° C. in an atmosphere of 5% CO₂ in air and 100% relative humidity (see e.g. Example 3). The presence of CO₂ would only be necessary to the extent that bicarbonate buffers are utilized, thus requiring ambient CO₂ for pH maintenance.

In one embodiment, osteopontin is combined with an embryo culture mixture. Such addition can improve the function of an embryo (i.e., improve the potential for normal development of the embryo). This potential of embryos is assessed by evaluating chromosome numbers, cell numbers, cytoskeleton formation and metabolic activity. Improved function means that the embryo has enhanced performance as assessed by one of these assays when treated with osteopontin under conditions described herein as compared to a control (i.e., no treatment with osteopontin). Preferably, the test of normal fertilization and function is embryo transfer and development to term.

In another embodiment, fertilized embryos or cultured fertilized embryos produced by the methods of the invention can be transferred to the reproductive tract of a surrogate animal. For example, fertilized embryos can be transferred to the reproductive tract of a gilt or sow. See e.g. Lai and Prather, Cloning & Stem Cells (2003) 5, 233-242.

Alternatively, the embryos might be cultured in vitro (see e.g. Im et al., Theriogenology. (2004) 61, 1125-1135), or in vivo (see e.g. Prather et al. Theriogenology (1991) 35, 1147-1151) prior to surgical (Cabot et al., Anim. Biotech. (2001) 12:(2) 205-214) or non-surgical embryo transfer to a suitable surrogate animal, for example a gilt or sow (see e.g. Martinez et al., Theriogenology (2003) 61, 137-146). Such embryos might be frozen or vitrified and thawed prior to the transfer (see e.g. Misumi et al., Theriogenology (2003) 60, 253-260).

After fertilization and before embryo transfer, the embryos can be cloned by nuclear transfer (see e.g. Prather et al., Biol. Reprod. (1989) 41:414-418) or made transgenic by a variety of methods including, but not limited to, pronuclear injection or viral transduction (see e.g. Wolf et al., Experimental Physiology (2000) 85, 615-625).

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

### Media

Unless otherwise stated, all chemicals used in this study were purchased from Sigma Chemical Co. (St. Louis, Mo.). Oocyte maturation medium was prepared as TCM 199 (Gibco BRL, 31100-76) supplemented with 0.1% PVA (w/v), 3.05 mM D-glucose, 0.91 mM sodium pyruvate, 75 μg/ml penicillin G, and 50 μg/ml streptomycin. The following were added fresh each time before use: 0.57 mM cysteine, 0.5 μg/ml luteinizing hormone (LH; Sigma, L-5269), 0.5 μg/ml follicle stimulating hormone (FSH; Sigma, F-2293), and 10 ng/ml epidermal growth factor (EGF; Sigma, E-4127). IVF medium was a modified Tris-buffered medium (mTBM) containing 2 mg/ml BSA and 2 mM caffeine. Osteopontin was diluted with PBS to a concentration of 0.001, 0.01, 0.1, or 1.0 μg/ml in mTBM. Sperm washing medium was Dulbecco phosphate-buffered saline (dPBS; Gibco) supplemented with 1 mg/ml BSA (pH 7.3). The culture medium for embryonic development was Porcine Zygote Medium-3 (PZM3, pH 7.3) medium supplemented with 3 mg/ml BSA.

### EXAMPLE 2

### Collection of Porcine Oocytes and In Vitro Maturation

Ovaries were collected from prepubertal gilts at a local abattoir and stored in 0.9% NaCl solution at 30-35° C. Cumulus-oocyte complexes (COCs) were aspirated from antral follicles (3-6 mm in diameter) with an 18-gauge needle fixed to a 10-ml disposable syringe. COCs with uniform cytoplasm and several layers of cumulus cells were selected and rinsed three times in TL-Hepes containing 0.1% (w/v) polyvinyl alcohol (PVA). Approximately 50-70 COCs were transferred into 500 μl IVM medium. The medium had been covered with mineral oil in a four-well Nunclon dish (Nunc, Roskilde, Denmark). The oocytes were matured for 4044 hr at 38.5° C., 5% CO₂ in air.

### EXAMPLE 3

### Production of Porcine Preimplantation Embryos by In Vitro Fertilization

Cumulus-free oocytes were washed three times in IVF medium. Approximately, 30-35 oocytes were transferred into 50 μl droplets of IVF medium covered with mineral oil that had been equilibrated for 40 hr at 38.5° C. in 5% CO₂ in air. The dishes were kept in a CO₂ incubator until sperm were added for insemination. For IVF, one 0.1 ml frozen semen pellet was thawed at 39° C. in 10 ml sperm washing medium. After washing 2 times by centrifugation (1900×g, 4 min), cryopreserved ejaculated spermatozoa were resuspended with fertilization medium to a concentration of 2×10⁶ cells/ml. Fifty μl of the sperm sample was added to the fertilization droplets containing the oocytes, giving a final sperm concentration of 1×10⁶ cells/ml. Osteopontin was added to the fertilization droplet at concentrations of 0.0, 0.001, 0.01, 0.1, or 1.0 μg/ml. Oocytes were co-incubated with the sperm for 6 h at 38.5° C. in an atmosphere of 5% CO₂ in air and 100% humidity. At six-hour postinsemination, oocytes were washed 3 times and cultured in 500 ul culture medium in 4-well Nunclon dishes at 38.5° C., in 5% CO₂ in air.

### EXAMPLE 4

### Evaluation of In Vitro Fertilization

At the end of the co-incubation period described above, oocytes were washed three times in development medium and transferred into 4-well Nunclon multidishes containing 500 μl of the same medium covered with 500 μl mineral oil and returned to the incubator for further development. After 18 h from the onset of IVF, half of the oocytes were transferred into one well of a 4-well plate, and the spermatozoa removed from the other half by vortexing for 1 min. After washing 3 times, the fertilized oocytes were transferred to the center of a glass microscope slide, covered with a cover slip and fixed with fresh fixing medium (25% (v/v) acetic acid in ethanol) for 72 h at room temperature. Orcein (1%, w/v) in 45% (v/v) acetic acid was added and the oocytes stained for 10 min at room temperature. The oocytes were then washed with 20% glycerol and 20% acetic acid in water. The slide was cleaned and then sealed with nail polish. Nuclear status (pronuclear, sperm head, sperm tail, MII chromosome, Pb1, Pb2) was then determined under a phase-contrast microscope at 400×.

The following effects of osteopontin on fertilization parameters were evaluated: penetration rate (number of oocytes penetrated by sperm/total number of oocytes inseminated), polyspermy rate (number of oocytes with >1 sperm/total number of oocytes penetrated), male pronuclear formation rate (number of oocytes with >1 male pronucleus/total number of oocytes penetrated), normal fertilization efficiency (number of oocytes with 1 male and 1 female pronucleus/total number of oocytes inseminated), and mean number of sperm penetrated per oocyte.

Experiments were repeated with 10 to 14 replications. Data (mean±SEM) were subjected to GLM of SAS followed by a protected LSD test. A p-value of less than 0.05 (p<0.05) was considered statistically significant.

Exemplary results demonstrated that osteopontin can decrease the incidence of polyspermy in pig IVF and result in an overall more efficient procedure (as a non-limiting example, approximately 44%) based on the number of oocytes inseminated. See e.g. Table 3. In these studies, the polyspermy rate decreased as the osteopontin concentrations increased: 0.01-1 μg/ml significantly reduced the polyspermy rate, compared to the control. See e.g. Table 1. Also, all levels of osteopontin significantly reduced the mean number of sperm in each oocyte as compared to the controls, and the effect was concentration dependent. At 0.01, 0.1 and 1.0 μg/ml osteopontin, the monospermy rate was increased as compared to the controls. See e.g. Table 2. The male pronucleus rate was decreased by the highest level of osteopontin as compared to the control. See e.g. Table 3. The overall fertilization rate (1 male and 1 female pronucleus per total number of oocytes inseminated) was elevated at 0.001 μg/ml osteopontin and significantly higher at 0.01 and 0.1 μg/ml osteopontin as compared to the control. See e.g. Table 3.

TABLE 1
Effect of OPN on polyspermy of pig oocytes during IVF.

| OPN | No. | No. Penetrated | | No. of sperm per |
|---|---|---|---|---|
| (μg/ml) | oocytes | oocytes | Polyspermy (%) | Oocyte (%) |
| 0.0 | 147 | 104 | 38.8 ± 3.1^{a} | 1.49 ± 0.06^{a} |
| 0.001 | 137 | 99 | 32.9 ± 3.1^{a,b} | 1.29 ± 0.06^{b} |
| 0.01 | 150 | 105 | 27.2 ± 3.1^{b,c} | 1.22 ± 0.06^{b,c} |
| 0.1 | 149 | 92 | 20.8 ± 3.1^{c,d} | 1.21 ± 0.06^{b,c} |
| 1 | 133 | 62 | 16.4 ± 3.1^{d} | 1.08 ± 0.06^{c} |

Within a column, values with different superscripts are significantly different (p < 0.05). Values are expressed as means ± SEM of ten replicates. Percentage polyspermy is calculated from the number of oocytes inseminated. Mean numbers of sperm are calculated from the number of penetrated oocytes.

TABLE 2
Effect of OPN on sperm penetration of pig oocytes during IVF.

| OPN | No. | No. Penetrated | Penetration | |
|---|---|---|---|---|
| (μg/ml) | Oocytes | oocytes | Rate (%) | Monospermy (%) |
| 0.0 | 147 | 104 | 70.8 ± 2.9^{a} | 45.7 ± 4.9^{a} |
| 0.001 | 137 | 99 | 74.6 ± 2.9^{a} | 57.5 ± 4.9^{a,b} |
| 0.01 | 150 | 105 | 73.7 ± 2.9^{a} | 63.3 ± 4.9^{b} |
| 0.1 | 149 | 92 | 67.7 ± 2.9^{a} | 69.0 ± 4.9^{b} |
| 1 | 133 | 62 | 49.3 ± 2.9^{b} | 70.2 ± 5.2^{b} |

Within a column, values with different superscripts are significantly different (p < 0.05). Values are expressed as means ± SEM of fourteen replicates. Percentage penetration is calculated from the number of oocytes inseminated. Percentage monospermy is calculated from the number of total penetrated oocytes.

TABLE 3
Effect of OPN on pronuclear formation of pig oocytes during IVF.

| OPN | No. | No. Penetrated | Male | Fertilization |
|---|---|---|---|---|
| (μg/ml) | oocytes | oocytes | Pronucleus (%) | Efficiency (%) |
| 0.0 | 147 | 104 | 59.5 ± 3.4^{a} | 31.6 ± 3.4^{c} |
| 0.001 | 137 | 99 | 65.7 ± 3.4^{a} | 41.6 ± 3.4^{a,b,c} |
| 0.01 | 150 | 105 | 63.2 ± 3.4^{a} | 42.6 ± 3.4^{a,b} |
| 0.1 | 149 | 92 | 57.1 ± 3.4^{a} | 44.6 ± 3.9^{a} |
| 1 | 133 | 62 | 40.6 ± 3.4^{b} | 32.9 ± 3.4^{b,c} |

Within a column, values with different superscripts are significantly different (p < 0.05). Values are expressed as means ± SEM of fourteen replicates. Percentage male pronucleus is calculated from the number of penetrated oocytes. Percentage normal fertilization are calculated from the number of oocytes inseminated.

### EXAMPLE 5

### Effect of Osteopontin on Sperm Function

To examine if the decreased polyspermy in vitro resulted from the changes in sperm function the effects of OPN on sperm motility, progressive motility, viability, and acrosome reaction were investigated.

Thawed sperm were incubated in mTBM containing 0, 0.1, or 1 μg/ml OPN for 2, 4, or 6 h at 38.5° C., in 5% CO₂ in air. The time immediately after thawing served as the 0 h group for all experiments. Porcine sperm motility and progressive motility for samples were analyzed at 0, 2, 4, and 6 h on a computer aided semen analyzer (Hamilton Thorne IVOS v 12.2c, Beverly, Mass.). Motility was defined as the percentage of spermatozoa that exhibited any movement of the sperm head. Progressive motility was defined as the percentage of spermatozoa that exhibited linear velocity of 45 μm/sec with a straightness of 45%.

Sperm viability was assessed in a fluorometric assay after being stained with propidium-iodide (PI, Sigma). Thawed semen samples were well mixed, transferred to 50 μl of IVF medium (pre-equilibrated with OPN overnight) containing various concentrations of OPN (0, 0.1 or 1 μg/ml) and co-incubated with spermatozoa for 0, 2, 4, or 6 h. At different points, PI (10 μg/ml) was added for 30 min in an incubator with CO₂ in the dark. After incubation, the sperm were transferred (10 μl) onto a glass slide, smeared, and mounted with an antifade reagent (ProLong®, Molecular Probes), covered with a glass cover slip, and sealed. Fluorescence was determined by using an epi-fluorescent microscope (Nikon, Tokyo, Japan). Sperm were observed at ×400 magnification, and at least 200 cells were evaluated per sample. Spermatozoa stained with PI were considered to have damaged membranes. The percentage of spermatozoa without PI staining is the sperm viability. Each group was replicated six times.

Results showed that the percentages of sperm motility, progressive motility, and viability decreased in all groups at 2 h after IVF, but were not different (p>0.05) between treatment groups.

Sperm acrosome reaction was investigated by staining with Alexa-PNA/DAPI, according to the procedure described by Sutovsky (Methods in Molec. Bio. (2003) 253, 59-77, Humana Press, Totowa, N.J.) and Katayama et al. (Human Reprod. (2002) 17, 2657-2664), with slight modifications. At 4 or 6 h after IVF, the oocytes were washed three times in 400 μl of dPBS-PVP medium in a prewarmed glass plate with 4-well dish on a slide warmer set to 37° C., and pipetted in and out (10 times) to remove loosely bound sperm. The oocytes were transferred into 400 μl of 2% formaldehyde in dPBS for 40 min at room 180 temperature (RT) for fixing. After fixation, the oocytes were washed twice in dPBS-PVP at RT, and then transferred to 0.1% triton X-100 in dPBS for 40 min at RT to permeabilize the oocytes. The oocytes were incubated in 0.4 μg/ml (1:500) Alexa-Fluo 488-PNA (Cat#L-21409, Molecular Probes) in 0.1% triton X-100 in dPBS for 40 min in the dark, and then transferred into 0.1% triton X-100 in dPBS for 5 min. The oocytes were transferred to a standard microscopy slide, in 8 μl mounting medium with DAPI (VECTASHIEID, H-1200, VECTOR) and covered with a cover slip that was then sealed with nail polish. Fluorescence was determined by using an epi-fluorescent microscope (Nikon, Tokyo, Japan). Sperm were observed at 1000× magnification, and 10 oocytes were evaluated per sample. The sperm around the ZP were counted according to Alexa-PNA and DAPI staining: spermatozoa were considered to be acrosome intact as determined by an Alexa-PNA-stained acrosome at top of the sperm with a DAPI nucleus. Sperm that had an acrosome area not stained with Alexa-PNA were considered to be acrosome reacted. The percentages of the number of the acrosome-reacted and the acrosome-intact in total spermatozoa around the ZP were examined. Each group was replicated 3 times.

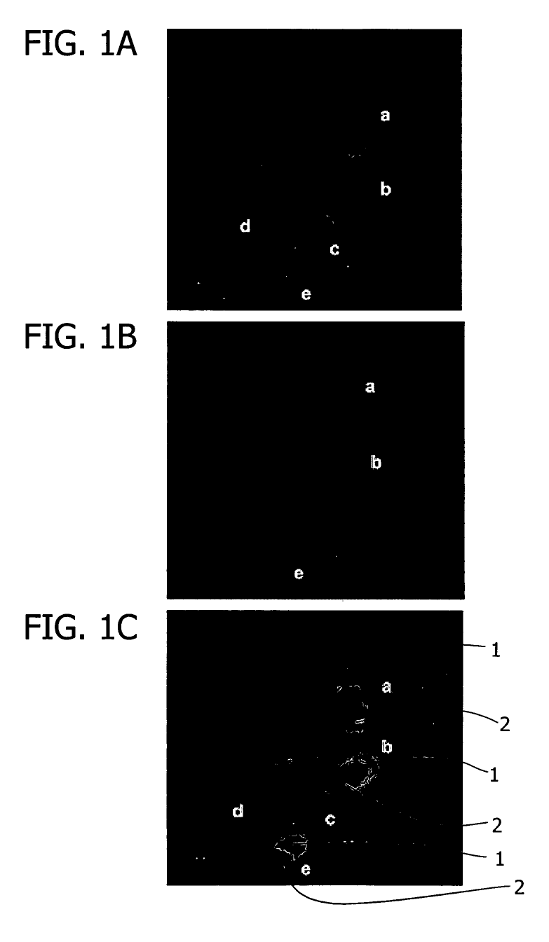

Results from observing the acrosome reaction with a fluorescence microscope at 4 h after IVF showed that the sperm bound to the ZP of the 1 μg/ml OPN treated oocytes had a higher rate of acrosome reaction as compared to 0 OPN (see e.g. FIG. 1). The lowest level of acrosome reaction was observed at 6 h after IVF with 0.1 μg/ml OPN (see e.g. Table 4).

TABLE 4
Acrosome reaction of sperm bound to the
zona pellucida at 4 or 6 h after IVF.

| | | | Total No. of | |
|---|---|---|---|---|
| OPN | Total No. of | Mean Sperm | Oocytes | Mean Sperm |
| (μg/ml) | Oocytes (4 hr) | Bound 4 hr (%) | (6 hr) | Bound 6 hr (%) |
| 0.0 | 30 | 75 ± 2.1^{a} | 28 | 97 ± 1.7^{a} |
| 0.1 | 30 | 76 ± 2.1^{a} | 26 | 87 ± 1.8^{b} |
| 1.0 | 20 | 87 ± 2.6^{b} | 27 | 95 ± 1.8^{a} |

Within a column, values with different superscripts are different (p < 0.05). Values are expressed as means ± SEM of six replicates.

### EXAMPLE 6

### Effect of Osteopontin on Oocyte Function

Zona pellucida solubility or ‘hardness’ was measured after exposure to 0.1% pronase. Cumulus-free oocytes matured in vitro were transferred to 50 μl of mTBM (pre-200 equilibrated with OPN overnight) containing various concentrations of OPN (0, 0.1 or 1 μg/ml) and were incubated for 6 h with/without spermatozoa at 39° C., 5% (v/v) CO₂ in air. Groups of 10 were used for the experiment without OPN (control) or with OPN (0.1, 1 μg/ml OPN). The oocytes were transferred into PBS and washed three times, and then transferred into 100 μl of 0.1% (w/v) pronase solution in dPBS. Zonae pellucidae were continuously observed for dissolution under an inverted microscope equipped with a warm plate at 37° C. The dissolution time of the ZP of each oocyte was registered as the time interval between placement of the samples in pronase solution and that when the ZP was no longer visible at a magnification of ×200. Each treatment was replicated six times. Results showed that the number of sperm bound per oocyte reduced as the concentration of OPN increased, but this was only significant (p<0.05) at 6 h after IVF (see e.g. Table 5).

TABLE 5
Effect of OPN on the number of sperm bound
to the zona pellucida during IVF.

| OPN | No. Sperm Bound | No. Sperm Bound |
|---|---|---|
| (μg/ml) | 4 Hr. | 6 Hr. |
| 0.0 | 26.3 ± 12.1 | 99.3 ± 7.9^{a} |
| 0.1 | 10.2 ± 12.1 | 64.9 ± 7.9^{b} |
| 1.0 | 3.4 ± 12.1 | 47.1 ± 7.9^{b} |

Within a column, values with different superscripts are different (p < 0.05). Values are expressed as means ± SEM of six replicates.

Sperm binding to the ZP was examined according to the methods described by Kouba et al. (Reproduction (2000) 63, 242-250), with slight modification. Cumulus-free oocytes matured in vitro were transferred to 50 μl of mTBM (pre-equilibrated with OPN overnight) containing OPN (0, 0.1 or 1 μg/ml) and co-incubated with spermatozoa for 4 or 6 hr. After fertilization, the oocytes were washed three times in 500 μl of mTBM and pipetted in and out (10 times) of a 215 pipette to remove loosely bound sperm. The oocytes were then placed into 50 μl drops of mTBM containing Hoescht 33342 (bis-Benzamide; 1.3 mg/ml) and incubated for 30 min at 39° C., 5% CO₂ in air in the dark. Oocytes were then washed twice in 300 μl of TLHepes-PVA, mounted, and the number of tightly bound sperm/zygote counted by using an epi-fluorescent microscope 400× (Nikon, Tokyo, Japan). Each treatment was replicated six times, with 10 oocytes counted from each replicate. Results showed that the duration in seconds required for ZP enzymatic digestion in the 0.1 μg/ml OPN treated groups was longer than the control group (p<0.05) after incubation with spermatozoa for 6 hours (see e.g. Table 6).

TABLE 6
The duration (seconds) for ZP solubility of oocytes exposed
to OPN and with or without spermatozoa at 6 hr after IVF.

| | Duration of ZP | Duration of ZP |
|---|---|---|
| | solubility with | solubility with OPN |
| OPN | OPN and sperm | and without sperm |
| (μg/ml) | (sec) | (sec) |
| 0.0 | 118 ± 8.9^{a} | 159.1 ± 8.9^{a} |
| 0.1 | 204.3 ± 8.9^{b} | 217.7 ± 8.9^{b} |
| 1.0 | 149.1 ± 8.9^{a} | 152.8 ± 8.8^{a} |

Within a column, values with different superscripts are different (p < 0.05). Values are expressed as means ± SEM of six replicates.

What is claimed is:

1. A method for in vitro fertilization comprising:
forming an in vitro fertilization mixture comprising osteopontin, an oocyte, and a sperm; and
allowing the sperm to fertilize the oocyte in the in vitro fertilization mixture.

2. The method of claim 1 further comprising the step of incubating the in vitro fertilization mixture for up to about 48 hours.

3. The method of claim 1 wherein the source of the oocyte is an oocyte mixture comprising osteopontin, an oocyte, and a buffer.

4. The method of claim 3 further comprising the step of incubating the oocyte mixture for more than 2 hours up to about 48 hours.

5. The method of claim 1 wherein the source of the sperm is a sperm mixture comprising osteopontin and sperm.

6. The method of claim 5 further comprising the step of incubating the sperm mixture for up to about 6 hours.

7. The method of claim 1 further comprising the step of culturing the fertilized oocyte to produce an embryo.

8. The method of claim 7 wherein culturing the fertilized oocyte to produce an embryo comprises forming an embryo culture mixture, wherein the embryo culture mixture comprises the fertilized oocyte, osteopontin, and a buffer.

9. The method of claim 7 further comprising the step of transferring the embryo to the reproductive tract of a surrogate animal.

10. The method of claim 7 further comprising the step of cloning the embryo by nuclear transfer.

11. The method of claim 10 further comprising the step of transferring the cloned embryo to the reproductive tract of a surrogate animal.

12. The method of claim 1 wherein osteopontin is present at about 0.001 to about 1.0 micrograms per milliliter of in vitro fertilization mixture.

13. The method of any one of claims 12 wherein osteopontin is present at about 0.01 to about 0.1 micrograms per milliliter of in vitro fertilization mixture.

14. The method of claim 1 wherein the mixture further comprises porcine oviduct-specific glycoprotein.

15. The method of claim 1 wherein polyspermy rate is reduced and the efficiency of in vitro fertilization is increased without substantially decreasing the penetration rate.

16. The method of claim 15 wherein the rate of polyspermy is less than about 36%.

17. The method of claim 16 wherein the rate of polyspermy is less than about 30%.

18. The method of claim 17 wherein the rate of polyspermy is less than about 25%.

19. The method of claim 18 wherein the rate of polyspermy is less than about 20%.

20. The method of claim 1 wherein the oocyte mixture comprises a porcine, human, bovine, canine, equine, ovine, avian, or rodent oocyte.

21. The method of claim 20 wherein the oocyte mixture comprises a porcine oocyte.

22. An aqueous mixture for in vitro fertilization comprising osteopontin, an oocyte, a sperm, and a buffer.

23. The aqueous mixture of claim 22 wherein the osteopontin is present at about 0.001 to about 1.0 micrograms per milliliter.

24. The aqueous mixture of claim 23 wherein the osteopontin is present at about 0.01 to about 0.1 micrograms per milliliter.
